(19) United States
(12) Patent Application Publication
(10) Pub. No.: US 2012/0288945 A1
(43) Pub. Date: Nov. 15, 2012

(54) System and method for determining contaminants in wastewater including shipboard bilge water

(76) Inventors: Bruce A. Russell, Chevy Chase, MD (US); Jean Shorett, Chevy Chase, MD (US)

(21) Appl. No.: 13/068,545

(22) Filed: May 13, 2011

(51) Int. Cl. G01N 33/92; B01L 3/00

(52) U.S. Cl. 436/63; 422/430

(57) ABSTRACT

A wastewater diagnostic system provides a housing that holds equipment to perform contaminant diagnostics in bilge water including shipboard bilge water. The wastewater diagnostic system utilizes a plurality of receptacles to house glass containers and reagents. The wastewater diagnostic system of the preferred embodiment includes shock-absorbing materials in the compartments to prevent movement and securely house glass containers and reagents. The wastewater diagnostic system further utilizes a flat work surface portion providing a test bed to conduct tests in non-laboratory environments. The system also provides methods to conduct tests to determine contaminants while utilizing the system.

### FIELD OF THE INVENTION

The present invention is directed to a system and method for diagnosing and determining the presence of contaminants in wastewater. The present invention is specifically directed to the determination of contaminants in bilge water located in maritime vessels, where the presence of contaminants may disrupt operation of vessel based Oily Water Separators (OWS). Additionally the present system and method may be utilized by untrained or minimally trained personnel while still providing consistent and accurate detection of the presence of contaminants in wastewater or bilge water samples.

### BACKGROUND OF THE INVENTION

The day-to-day operation of maritime vessels results in the formation of bilge water; water, oil, emulsified fluids and small particles that collect at the bottom of the vessel. Bilge water is usually disposed at sea but must first meet international standards for disposal prior to being discharged overboard. The International Convention for the Prevention of Pollution from Ships (MARPOL), Annex I, prohibits ships from discharging water with more than 15 parts per million of oil. Bilge water is often contaminated with oil and other byproducts of shipboard equipment and activities. Ships use Oily Water Separators (OWS) to remove oil from bilge water. However, many OWS malfunction particularly when bilge water contains materials other than oil and water. Materials such as soot, detergent, sewage, solvents, bacterial and microbial decomposition can lead to OWS malfunction and thereby prevent the discharge bilge water complying with MARPOL.

Ship owners, operators, and crews have long recognized that OWS are prone to malfunction. If a malfunction is unresolved, a ship may need to incur the cost and delay of service calls or pumping the entire bilge water as hazardous waste at port. Depending on the size of the ship, ship owners, operators and crew may have to pump thousands of gallons of bilge water, which could cost them tens and thousands of dollars. A malfunctioning OWS can also lead to illegal discharge of non-compliant bilge water, which may result in local, U.S. and international criminal and/or civil sanctions against culpable parties. Most importantly, the discharge of non-compliant bilge water, will likely result in serious short and long term ecological damage to marine environments. Thus, there is a need for a system and method for early and consistent testing of bilge water.

To address the issues of malfunctioning OWS, the International Maritime Organization (IMO) published IMO MEPC.1/Circ.677, “Guide To Diagnosing Contaminants In Oily Bilge Water To Maintain, Operate and Troubleshoot Bilge Water Treatment Systems” identifying seven tests that may be separately or collectively be utilized to determine the presence of materials not limited to oil in bilge water that can cause OWS malfunctions. Beyond enhancing the ability of crews and otheres to diagnose and address single or on-going OWS malfunctions, the Guide provides guidance to reduce or avoid OWS malfunctions on a continuous or ongoing basis. The continuous or periodic approach includes routine testing and monitoring of bilgewater to identify contaminants before OWS malfunctions occur. It also provided guidance on options for shipboard operation, maintenance practices, and structural changes as corrective actions to remove or reduce identified contamination sources, reducing future risks of bilgewater contamination and OWS failure. However, all IMO guides are not prescriptive and have no specific or practical means of conducting qualitative analysis in limited spaces or non-lab environments. The Guide also assumes that the performer of the tests will have basic or advanced testing knowledge.

In reality, due to space restrictions, motion artifacts including the 3-D motion of a vessel's yaw, pitch, and roll, contamination issues, costs and expenditures, full-scale tests in shipboard environs are impractical or implausible. Additionally, ships do not generally include personnel having testing expertise, thereby requiring untrained and minimally trained personnel to perform such testing duties. Thus, a lack of adequate and explicit instructions, proper equipment and the ability to make rapid qualitative analyses while a vessel is underway have resulted in inconsistent treatment of these waters that raise risks of illegal discharge and ecological damage.

There exist many technologies that concern themselves with the filtration or purification of bilge water using OWS technology. Exemplary of such include U.S. Pat. No. 7,890,226; U.S. Pat. No. 7,661,380; U.S. Pat. No. 7,491,320; U.S. Pat. No. 4,886,607; U.S. Patent Publication 2010/0282687; U.S. Patent Publication 2010/0213132; U.S. Patent Publication 2009/0050042; and U.S. Patent 2007/0068874. However, none of them address issues relating to determining the presence of contaminants in wastewater including bilge water. As a qualitative analytical means, they do not provide a system or method of low-tech testing that is capable of being performed within the confines of marine vessels or other non-lab environments and by staff with little technical expertise.

Further, none of the prior art on systems in the form of a portable housing address use in diagnosing and determining the presence of contaminants in bilge water. U.S. Pat. No. 5,025,920, for example, describes of a kit having a plurality of compartments for the purpose of gathering body and body fluid evidence. U.S. Pat. No. 4,195,059 describes of a kit for the purpose of performing chemical tests. U.S. Pat. No. 4,828,113 describes of a kit having a plurality of recesses for holding instruments used for dental treatment. U.S. Pat. No. 4,595,102 describes of a kit having a plurality of recesses that house tools to conduct medical procedures. Futher, U.S. Design Pat. 282,399 show as a “water analysis laboratory” and U.S. Design Pat. 297,166 shows a “reagent cassette.” These aforementioned prior art documents differ from the present invention because none of them show or describe a system and method for diagnosing and determining the presence of contaminants in bilge water.

For the reasons given above, there is, therefore, a need for a system and method that allows untrained or minimally trained personnel to successfully perform tests described in IMO MEPC.1/Circ.677 in shipboard and other non-laboratory environments. The present invention provides a system and method to meet these needs as described herein below.

### SUMMARY OF THE INVENTION

It is therefore, the objective of the present invention to disclose a system and method for diagnosing contaminants in wastewater.

It is another objective of the present invention to disclose a system that utilizes a housing having an open and closed position.

It is another objective of the present invention to disclose a system that utilizes a housing having a first and second interior section.

It is another objective of the present invention to disclose a system that utilizes a housing having a first and second interior section with a plurality of receptacles having stability portions, including but not limited to beakers, reagent bottles, and tools secured in compartments, while the system is in the open position, and allowing for tests to be administered to the wastewater sample without removing beakers or reagents from compartments, providing a stable work surface.

It is another objective of the present invention to disclose a system that utilizes a first interior section that provides a flat work surface portion.

It is another objective of the present invention to disclose a system that utilizes a first interior section that provides a housing compartment to house testing implements.

It is another objective of the present invention to disclose a system that utilizes a second interior section that provides one partially covered compartment to hold instruction materials.

It is another objective of the present invention to disclose a system that utilizes a second interior section that provides indented portions that align with the receptacles in first interior section when in closed position.

It is another objective of the present invention to disclose a system that utilizes housing, receptacles, indented portions, and stability portions to provide a stable, leakproof holder and carrier.

It is another objective of the present invention to disclose a system for diagnosing contaminants in wastewater including bilge water.

It is another objective of the present invention to disclose a system and method utilizing at least one glass container and at least one reagent to determine the presence of non-emulsified oil, emulsified oil, alkaline solvents, iron particles, bacterial and microbial decomposition, soot and observing color and opacity as a means of determining the presence of contaminants in bilge water.

### BRIEF DESCRIPTION OF THE DRAWINGS

FIG. 1 is a PLAN VIEW OF THE SYSTEM IN THE FIRST OPEN POSITION.

FIG. 2 is a CROSS-SECTIONAL VIEW TAKEN ALONG LINE A-A′ OF FIG. 1.

FIG. 3 is a PERSPECTIVE VIEW OF THE SYSTEM IN THE SECOND CLOSED POSITION.

### DETAILED DESCRIPTION OF THE EMBODIMENTS

The present invention addresses a long-felt need for providing an efficacious test system and method for detecting contaminants in wastewater, specifically bilge water in ships. Due to space restrictions, motion artifacts, contamination issues, costs and expenditures, full-scale tests in shipboard and non-laboratory environs are impractical or implausible. Additionally, ships do not generally include personnel having testing expertise, thereby requiring untrained and minimally trained personnel to perform such testing duties. Thus, the present invention overcomes prior problems associated with testing real-time in situ qualitative testing of wastewater including bilge water. The present system and method provides cost effective, rapid, efficacious and consistent results regardless of the expertise of the person performing these tests. It further provides easily accessible guidance on pro-active options for improved Oily Water Separators (OWS) performance such as bilgewater monitoring and corrective actions for identified contaminants. For this reason, the present invention is applicable for use beyond ships and other vessels utilizing an OWS. OWS technology is used in a broad range of manufacturing, off-shore drilling and other energy production operations. The present invention provides the same cost-effective, rapid, efficacious, and consistent results in these circumstances. However, it is important to distinguish the present invention from those directed to diagnosing or responding to oil spills or other instances where the presence of contaminants would not hinder direct removal of the subject oil. Further, the present invention is not directed to diagnosing or responding to chemical or sewage discharges where the ability of an OWS to separate oil from water is not a central concern.

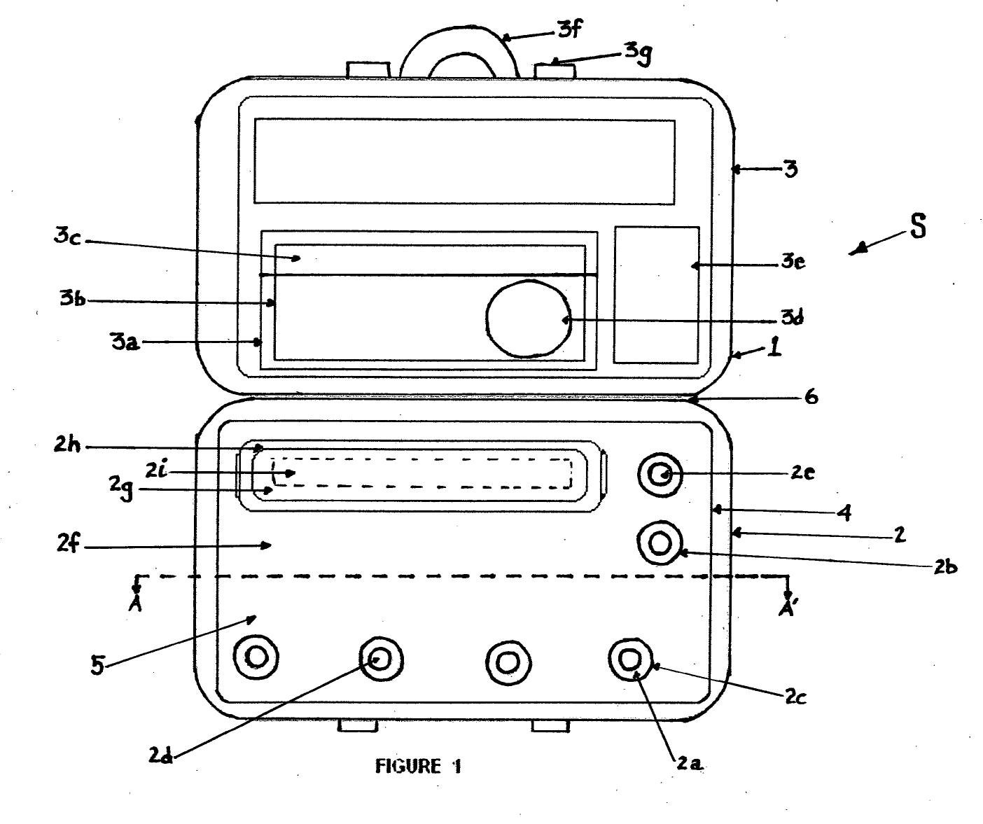

Referring to FIG. 1, the present invention discloses a system S that includes a housing 1 having a first interior section 2 and a second interior section 3. The first interior section 2, and second interior section 3 are formed using a shell 4 and an inner material 5. Hinges 6 connect the first interior section 2 and the second interior section 3 to each other.

The first interior section 2 includes a plurality of glass container receptacles 2a and a plurality of reagents receptacles 2b, preferably positioned along the edges of first interior section 2. Positioned within receptacles 2a and 2b are a shock absorbing material 2c such that glass containers 2d and reagents 2e are removably positioned within receptacles 2a and 2b respectively and in a manner that provides a frictional fit and further in a manner that is resistant to breakage, vibrations and movement of glass containers 2d and reagents 2e. The shock absorbing materials 2c include but are not limited to rigid foam. The combination of the receptacles 2a and 2b and the shock absorbing material 2c provide a stable work platform where tests may be conducted without having to remove the glass containers 2d and reagents 2e. As will be understood by one of ordinary skill in the art, glass containers 2d and reagents 2e are those which may be utilized to perform the tests as identified by the present invention. Notably, glass containers 2d, include but are not limited to lab grade, heat resistant glass beakers and cylinders. In a preferred embodiment, the reagents 2e are provided with color coding and/or incremental numbering commensurate with the test being performed.

In a preferred embodiment, one of the reagents includes but is not limited to 10% solution of concentrated sulfuric acid or a non-organic acid in type and concentration that will not chemically react with oil, but will cause emulsified oil to break out of emulsion. One of the reagents may also include but is not limited to 20% concentration of citric acid, or an organic acid that reacts with iron and iron oxides to cause effervescence.

The first interior section 2 is also constructed to provide a flat work surface portion 2f, thereby providing a stable flat surface where the personnel may conduct the tests described in the present invention. The flat work surface portion 2f is surrounded by the receptacles 2a, 2b and housing compartment 2g. The housing compartment 2g has a transparent cover 2h, in which testing implements 2i are housed.

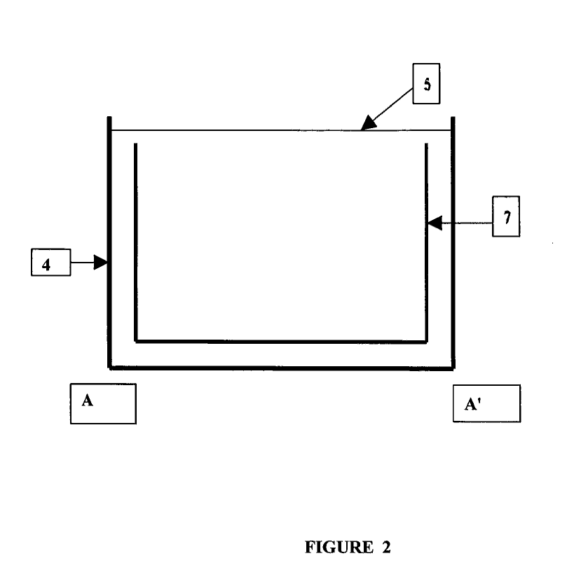

As shown in FIG. 2, the first interior section 2 and second interior section 3, are formed using a shell 4 and an inner material 5. In a preferred embodiment, there exists a space 7 between the shell 4 and the interior material 5. This is to provide additional protection to the glass containers 2d and reagents 2e if the system S is dropped or an application of an external force to the system S.

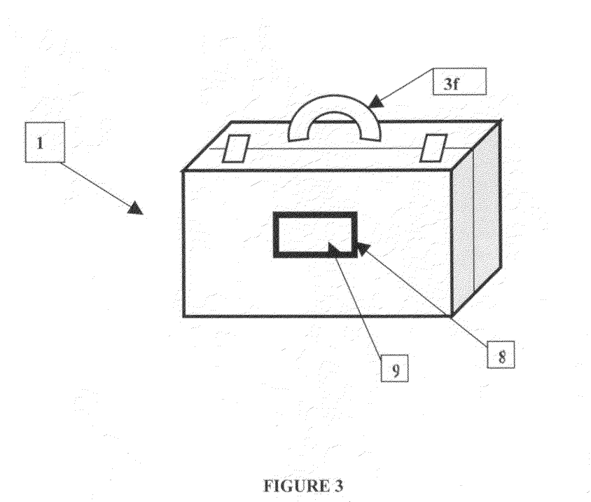

As shown in FIG. 3, the transparent cover 2h prevents testing implements 2i from falling out of the housing compartment 2g and moving about in the housing 1 when it is in its closed position and allows for easy storage of the testing implements 2i. The testing implements 2i include but are not limited to tweezers, glass stirrer and pH strips.

In a preferred embodiment, the first interior section 2 is one of two halves of the housing that is identical in size that form the housing. The second interior section 3 constitutes the other half of the housing 1. The shell 4 is fabricated from textured heavy-grade plastic to protect glass containers 2d and reagents 2e and other elements of the housing 1 from breaking or being dislodged by outside forces.

In another preferred embodiment, the transparent cover 2h is capable of being in both an open or closed position. The transparent cover 2h may include a sliding mechanism or a door that allows the cover to be moved from an open to a closed position (not shown).

The second interior section 3 includes a compartment 3a that is partially covered by a stationary transparent material 3b. Compartment 3a is used to house instructional/informational materials including but not limited to a user's guide 3c and an electronic storage media 3d for providing information/instructions in electronic form. The second interior section 3 further includes a number of indentations 3e, positioned such that indentations 3e align with glass container receptacles 2a and reagent receptacles 2b in first interior section 2. Indentations 3e provide clearance for glass containers 2c and reagents 2e when the housing 1 is in its closed position (see FIG. 3) as well as preventing dislodging and movement of glass containers 2c and reagents 2e in situations when they are removed from their respective receptacles 2a, 2c. A handle 3f is attached to the outside facing wall of the second interior section 3 opposite of where the hinges 6 are located. Latches 3g that secure the housing in its closed position are also attached on the same wall, on either side of the handle 3f.

In a preferred embodiment, the electronic storage media 3d also contains a test log, in addition to instructions, allowing for accurate and convenient record keeping of previous test results. Further, the electronic storage media 3d includes but is not limited to compact discs.

As shown in FIG. 3, the housing 1 in the closed position provides a stable, leakproof holder and carrier for the system. The housing 1 includes an indentation 8 for a label 9 on one of the outer walls of either the first interior section 2 or the second interior section 3.

The system S is utilized for determining and detecting for the presence of contaminants in wastewater. In a preferred embodiment, the system S is used to detect, distinguish and determine for the presence of contaminants in bilge water. The contaminants include, but are not limited to non-emulsified oil, emulsified oil, detergents and alkaline solvents, iron particles, bacterial and microbial decomposition and soot. The user's guide 3c provide test methods that encompass materials provided in system S and discussed above.

The following method in accordance with the present invention is utilized. In a preferred embodiment the methods are utilized in testing bilge water in environments where OWS are utilized. In another preferred embodiment the system and method of the present invention may be utilized in a batch test manner, to identify specific contaminants. In yet another preferred embodiment, the present invention may be utilized in a periodic (here, periodic includes continuous processes) manner to evaluate the condition of bidge water. By evaluating the bilge water, the imminent and/or future malfunction or failure of the OWS, due to contamination, may be determined and corrective actions taken to reduce the risk of failure However, as understood by one of ordinary skill, it is within the scope of the present invention to include other bilge water and wastewater testing methods not specifically disclosed. The order in which tests to determine the presence of contaminants can be varied and does not have to follow the order described below. The system S is designed to conduct tests in sequence to minimize the number of samples drawn and glass containers 2c to be used.

System S may be utilized to determine and detect for the presence of non-emulsified oil in wastewater, particularly bilge water:

1. Pour 40 ml of the bilge water sample into a beaker.

2. Allow sample to sit for 10 minutes.

3. Look for oil at top of the sample in the beaker.

4. If oil is present, it may be the result of one or more problems. See Section 2 of Circ 677/Annexes 5 & 6 for possible causes of the problem(s). Based on the likely causes, see Section 4 of Annexes 5 & 6 for corrective actions. Check Section 5 of Annexes 5 & 6 for preventive measures to help avoid the problem(s) in the future.

5. Mark results in the Bilge Water Test Log.

System S may be utilized to determine and detect for the presence of contaminants in wastewater, particularly bilge water using color and opacity:

1. Pour 40 ml of a bilge water sample into a beaker—or continue using the sample from Test #1.

2. Observe color/opacity. A cloudy, black or colored sample can be the result of one or more problems.

3. If the sample is cloudy, go to Circ. 677/Appendix II. If the sample is black or colored, see Section 2 of Circ. 677/Annexes 3, 4, & 5 for possible causes of the problem(s). Based on the likely causes, see Section 4 of Annexes 3, 4, & 5 for corrective actions. Check Section 5 of Annexes 3, 4, & 5 for preventive measures to help avoid this problem in the future.

4. Mark results in the Bilge Water Test Log.

System S may be utilized to determine and detect for the presence of emulsified oil in wastewater, particularly bilge water:

1. Pour 40 ml of the bilge water sample into a beaker.

2. Add three drops of Reagent 1 (Black Label). Put the top back on the bottle & close it tightly—or the reagents can leak. Stir sample easily for 30 seconds with the glass stirring rod.

3. Allow sample to sit for 10 minutes.

4. Look for oil at top of the sample in the beaker.

5. If oil is present, go to Section 4 of Circ. 677/Annex 3 for corrective actions. Check Section 2 of Annex 3 for causes of this problem and see Section 5 of Annex 3 for preventive measures to help avoid this problem in the future.

6. Mark results in the Bilge Water Test Log.

System S may be utilized to determine and detect for the presence of detergents and alkaline solvents in wastewater, particularly bilge water:

1. Pour 40 ml of the bilge water sample into a beaker. Fill a separate beaker with clean water.

2. Evaporate the bilge sample by placing it on a hot surface or using the optional hot plate. Note: To speed up evaporation, the sample can be reduced to 10 ml—but for best results use a 40 ml sample.

3. When the bilge water sample has evaporated, use tweezers to take 1 pH strip out of its package. Put the lid back on the pack immediately and make sure the lid is tight—or strips can become contaminated and useless.

4. Hold the pH strip with tweezers and dip it in the clean water. Swipe the pH strip in the evaporated residue of the bilge water.

5. Observe pH strip color. If the strip turns blue, go to Section 4 of Circ. 677 Annex 2 and Appendix II for corrective actions. Check Section 2 of Annex 2 for causes of this problem and see Section 5 of Annex 2 for preventive measures to help avoid this problem in the future.

6. Mark results in the Bilge Water Test Log.

System S may be utilized to determine and detect for the presence of iron particles in wastewater, particularly bilge water.

1. Pour 40 ml of a bilge water sample into a beaker

2. Add three drops of Reagent 2 (Red label). Put the top back on the bottle & close top tightly—or reagent can leak. Stir the sample easily for 3-4 circles with the glass stirring rod.

3. Observe the beaker. If gassing occurs and color changes, iron particles are present.

4. If iron is present go to Section 4 of Circ. 677/Annex 4 for corrective actions. Check Annex 4 Section 2 for causes of this problem and Section 5 for preventive measures.

5. Mark results in the Bilge Water Test Log.

System S may be utilized to determine and detect for the presence of bacterial and microbial decomposition in wastewater, particularly bilge water.

1. Pour 40 ml of a bilge water sample into a beaker.

2. Add three drops of Reagent 2 (Red label). Put the top back on the bottle & close it tightly—or reagent can spill. Stir sample easily for 30 seconds with the glass stirring rod.

3. Observe beaker. If particles collect at bottom, bacteria/microbial composition are present.

4. If bacteria/microbial composition are present, go to Section 4 of Circ. 677/Annex 4 for corrective actions. Check Annex 4 Section 2 for causes of this problem and Section 5 for preventive measures.

5. Mark results in the Bilge Water Test Log.

System S may be utilized to determine and detect for the presence of soot in wastewater, particularly bilge water.

1. Pour 40 ml of a bilge water sample into a beaker.

2. Add three drops of Reagent 2 (Red Label) (Note: Do not add more reagent if you are using materials from Test #5 & #6). Stir easily for 30 seconds with the glass stirring rod.

3. Evaporate sample by placing on a hot surface or using the optional hot plate.

4. If black residue is observed soot is present. If soot is present, go to Section 4 of Circ. 677/Annexes 3 & 4, and Appendix II for corrective actions. Check Annex 4 Section 2 of for causes of this problem and see Section 5 of Annex 4 for preventive measures to help avoid this problem in the future.

5. Mark results in the Bilge Water Test Log.

What is claimed is:

1. A system for diagnosing contaminants in wastewater comprising:
a housing having a handle and a rigid structure, said housing constructed so as to have a first open position and a second closed position, said housing further comprising a first and second interior sections connected to each other utilizing hinges, said first and second interior sections further constructed such that said first and second interior sections are accessible in said open position;
wherein said first interior section is constructed so as to provide a plurality of receptacles, said receptacles further constructed so as to include stability portions;
wherein said first interior section further constructed so as to provide a flat work surface portion;
wherein said first interior section further constructed with a housing compartment as to house testing implements;
wherein said second interior section constructed so as to provide at least one partially covered compartment constructed so as to hold instructional materials, said second interior section further constructed so as to provide indented portions, said indented portions constructed so as to align with said compartments of said first interior sections when said rigid structure is in said closed position; and
said housing, receptacles and said stability portions providing a stable, leakproof holder and carrier for said system.

2. The system as recited in claim 1, wherein said housing further comprises an outer surface having a handle positioned thereon said housing constructed so as to provide a rigid structure wherein said handle and rigid structure are constructed so as to provide stability and contamination resistant features to said system.

3. The system as recited in claim 2, further comprising said stability portions having shock absorbing means.

4. The system as recited in claim 3, wherein said plurality of receptacles in said first interior section further comprising glass containers and at least one reagent, wherein said glass containers and said at least one reagent are constructed so as to be individually and removably placed in said plurality of receptacles.

5. The system as recited in claim 4, wherein said interior sections further comprise of a shell and an inner material.

6. The system as recited in claim 5, wherein said wastewater consists essentially of bilge water, said system further constructed so as to determine the presence of said contaminants in said bilge water.

7. The system as recited in claim 6, wherein said contaminants comprise:
non-emulsified oil, emulsified oil, detergents and alkaline solvents, iron particles, bacterial and microbial decomposition components and soot.

8. A system for diagnosing contaminants in wastewater comprising:
wherein said wastewater consists essentially of bilge water, said system further constructed so as to determine the presence of said contaminants in said bilge water;
a housing having a handle and a rigid structure, said housing constructed so as to have a first open position and a second closed position, said housing further comprising a first and second interior sections connected to each other utilizing hinges, said first and second interior sections further constructed such that said first and second interior sections are accessible in said open position;
wherein said first interior section is constructed so as to provide a plurality of receptacles, said receptacles further constructed so as to provide stability portions;
wherein said first interior section further constructed so as to provide a flat work surface portion;
wherein said first interior section further constructed with a housing compartment as to house testing implements;
wherein said second interior section constructed so as to provide at least one partially covered compartment constructed so as to hold instructional materials, said second interior section further constructed so as to provide indented portions, said indented portions constructed so as to align with said compartments of said first interior sections when said rigid structure is in said closed position;
wherein said housing, receptacles, indented portions, and stability portions providing a stable, leakproof holder and carrier for said system;
wherein said housing further comprises an outer surface having a handle positioned thereon said housing constructed so as to provide a rigid structure wherein said handle and rigid structure are constructed so as to provide stability and contamination resistant features to said system;
wherein said stability portions having shock absorbing means;
wherein said plurality of compartments in said first interior section further comprising glass containers and reagent receptacles, wherein said glass containers and said reagent receptacles are constructed so as to be individually and removably placed in said compartments;
wherein said interior sections further comprise of a shell and an inner material; and
wherein said contaminants comprise non-emulsified oil, emulsified oil, detergents and alkaline solvents, iron particles, bacterial and microbial decomposition components and soot.

9. A method of diagnosing and testing a plurality of contaminants in wastewater comprising:
Utilizing a diagnostic system having a holder comprising a plurality of compartments, said compartments housing a plurality of reagents utilizing said reagents for determining said plurality of contaminants in wastewater;
Utilizing at least one of said glass containers and one of said plurality of reagents and determining the presence of non-emulsified oil as one of said plurality of contaminants in wastewater;
Utilizing at least one of said glass containers and one of said plurality of reagents and determining the presence of emulsified oil as one of said plurality of contaminant in wastewater;
Utilizing at least one of said glass containers and one of said plurality of reagents and determining the presence of detergents and alkaline solvents as one of said plurality of contaminant in wastewater;
Utilizing at least one of said glass containers and one of said plurality of reagents and determining the presence of iron particles as one of said plurality of contaminants;
Utilizing at least one of said glass containers and one of said plurality of reagents and determining the presence and concentration of bacterial and microbial decomposition as one of said plurality of contaminants;
Utilizing at least one of said glass containers and one of said plurality of reagents and determining the presence of soot as one of said plurality of contaminants; and
Utilizing at least one of said glass containers and one of said plurality of reagents and determining color and opacity as a means of determining the presence of one or said plurality of contaminants in said wastewater.

10. The method as recited in claim 9, wherein said wastewater consists essentially of bilge water.

11. The method as recited in claim 10, wherein said determination of presence of non-emulsified oil further comprising the steps of:
(a) pouring 40 milliliters of bilge water sample into one of said glass containers;
(b) resting said sample for 10 minutes; and
(c) observing for said non-emulsified oil in said bilge water.

12. The method as recited in claim 11, wherein said determination of presence of emulsified oil further comprising the steps of:
(a) pouring 40 milliliters of bilge water sample into one of said glass containers;
(b) resting said sample for 10 minutes; and
(c) observing for said emulsified oil in said bilge water sample of (a).

13. The method as recited in claim 12, wherein said determination of presence of detergents and alkaline solvents further comprising the steps of:
(a) pouring 40 milliliters of bilge water sample into one of said glass containers;
(b) pouring clean water in another one of said glass containers;
(c) evaporating said bilge water sample;
(d) utilizing said clean water and colorometric pH testing means and determining pH of said bilge water sample of (a).

14. The method as recited in claim 13, wherein said determination of presence of iron particles further comprising the steps of:
(a) pouring 40 milliliters of bilge water sample into one of said glass containers;
(b) adding one of said reagents to said bilge water sample; and
(c) observing release of gases and change of color in said bilge water sample of (a).

15. The method as recited in claim 14, wherein said determination of presence of bacterial and microbial decomposition further comprising the steps of:
(a) pouring 40 milliliters of bilge water sample into one of said glass containers;
(b) adding one of said reagents to said bilge water sample; and
(c) observing for precipitation of matter indicative of bacterial and microbial decomposition in said bilge water sample of (a).

16. The method as recited in claim 15, wherein said determination of presence of soot further comprising the steps of:
(a) pouring 40 milliliters of bilge water sample into one of said glass containers;
(b) adding one of said reagents to said bilge water sample, stirring said bilge water sample and reagent solution;
(c) evaporating said solution; and
(d) observing for black residue in said bilge water sample of (a).

17. The method as recited in claim 16, wherein said determination of presence of one of said plurality of contaminants in said bilge water includes pouring 40 milliliters of bilge water sample into one of said glass containers, and observing visual alterations of said bilge water sample.

18. The method as recited in claim 17, wherein said visual alterations comprise observing colorometic alterations and opacity alterations.

19. The method as recited in claim 18, wherein the reagent is selected from the group comprising a 10% solution of sulfuric acid, inorganic acids that do not chemically react with oil, 20% concentration of citric acid, and organic acid that reacts with iron and iron oxides.

20. The method as recited in claim 19 and monitoring said at least one contaminants in a batch or periodic manner and determining whether an oily water separator is malfunctioning and further determining whether said separator is approaching failure.
